(19) United States
(12) Patent Application Publication
(10) Pub. No.: US 2016/0015509 A1
(43) Pub. Date: Jan. 21, 2016

(54) IMPLANTABLE STENT

(71) Applicant: Ardle Tomás McDonough, Galway (IE)

(72) Inventor: Ardle Tomás McDonough, Galway (IE)

(21) Appl. No.: 14/336,822

(22) Filed: Jul. 21, 2014

(51) Int. Cl. A61F 2/04

(52) U.S. Cl. CPC A61F 2/04; A61F 2002/047

(57) ABSTRACT

The invention is a stent designed for indwelling in a body, where its purpose would be to assist in the drainage of liquid from one part of the body to another. The stent is composed of a material that would typically be coiled to form a cylindrical shape along the length of the stent. Each end of the stent would typically be shaped to form a looped pigtail to prevent migration in the vessel. The stent can be used for minimally invasive procedures or alternatively, it could be placed percutaneously. This stent could be used in various parts of the body, such as the ureter, urethra, bile duct, liver, pancreas, vascular system and neurovascular system.

### REFERENCES CITED

### U.S. Patent Documents

U.S. Pat. No. 7,550,012 Jun. 23, 2009 Lavelle

### U.S. Patent Documents

U.S. Pat. No. 4,813,925 Mar. 21, 1989 Anderson & Maerzke

### TECHNICAL FIELD

The technical field of this invention is an implantable medical device. This invention is particularly suitable for use in veterinary or paediatric urological procedures.

### BACKGROUND OF THE INVENTION

The placement of urology stents via minimally invasive techniques has continued to increase incrementally over the last decade. Both metal and plastic stents of various designs are being placed.

A ureteral stent, for example, can be placed in certain circumstances to allow urine to drain from the kidney to the bladder and out of the body, when urine cannot flow through the ureter with ease.

Recent advances in veterinary medicine have also enabled the placement of urology stents in animals. However, many or all of the urology stents being placed in veterinary medicine at present are plastic stents. These products are generally susceptible to higher levels of encrustation than metal stents because of a reaction between the plastic stent and bacteria in the urinary system. The encrustation can make the stents difficult to remove, which is obviously problematic. For this reason, the recommended indwell duration for human use of these plastic stents is only eight weeks, which is rarely suitable for the intended use in animals.

Another shortcoming with plastic stents of smaller diameters is low radial strength. This is particularly problematic when there is a tight stricture, a stone that cannot be easily removed and where the ureter of the patient is between 0.3 mm and 1.0 mm in diameter. In some cases, the stent is unable to withstand the compressive forces exerted on it and it malfunctions (“High failure rate of indwelling ureteral stents in patients with extrinsic obstructions”, Docimo S G, DeWolf W C, 1989).

A further issue is an inability to produce smaller diameter stents with current stent designs and stent kits. Inserting stents that exceed the size of the vessel can cause unnecessary and irreversible dilation of the vessel. This is an issue where vessels are smaller than any stent available, such as in the ureters or pancreatic ducts of smaller animals, such as cats or small dogs. The result of placing stents that exceed the size of the vessel is that the stent can never be removed, since the vessel would collapse.

Furthermore, since the size of current stents is larger than the vessel for which they are intended, there is difficulty in placing the current stents. Hospital procedures and duration of anaesthesia are therefore longer than necessary.

It would not be possible to use any stent or stent kit previously invented to solve the problems described above. The stent and stent kit listed in this patent claim is a solution to these issues.

With regard to metal ureteral stents, the stents and stent kits listed in the prior art quoted in this patent claim would not work for smaller stents.

The coils in patent claim U.S. Pat. No. 4,813,925 are coiled helically, but are not touching, which reduces radial strength. The wire is a single wire only, which also reduces radial strength and which reduces holding strength in the looped ends of the stent. There is therefore a higher chance of deformation and inability to maintain its place in a stent such as this in smaller versions of stent use, where wires are akin to thread used in common sewing. The use of hollow wire to allow for a guidewire to be inserted into the wire to place the stent would also not be possible in smaller wire dimensions.

The stent kit in patent number U.S. Pat. No. 7,550,012 uses a hollow wire, which would not be feasible for smaller stents as wire dimensions decrease, due to the loss of wall strength in the wire. Similarly, the delivery system would lack sufficient compressive strength to place a stent in smaller dimensions, which is the reason for the innovation of a mandrel of high compressive strength in the stent inserter in this invention.

The dome-shaped caps and cannula used in the patent number U.S. Pat. No. 7,550,012 would most likely prevent the coil stretching excessively on removal. The stent in this invention would uses a three-hilar coil, laser-welded at the tip to produce a coil that would remain compact on removal and that would be cheaper and more efficient in terms of resources to produce than the stent in patent number U.S. Pat. No. 7,550,012.

### BRIEF SUMMARY OF THE INVENTION

The unique design of this stent and stent kit allows for the creation of smaller, better functioning stents than are currently capable of being produced.

This stent possesses a suitable radial strength to enable bodily vessels to function regardless of any type of unwanted forces imposed on and in those vessels. It is composed of a material that provides for the stent remaining in place for an extended indwell duration of at least one year and being removed at that point without issue.

It can also be produced in a range of sizes to fit all ureters, thereby eliminating the possibility of damage and trauma to the patient. This is of obvious benefit to both physicians placing the stents and to patients.

This invention has clear uses as a stent to assist urine flow in the ureter and the urethra, where issues have occurred to stem urine flow, but it can additionally be used in other parts of the body, as outlined in the abstract.

A good system of delivering the stent to its intended place of use is key in a stent kit design. Without a functioning delivery system, the stent would have to be placed percutaneously, which should be avoided if possible. Percutaneous placement involves cutting open the body of the patient to place the stent, which exposes the body to possible infection and which causes pain and slower recovery. While there are a number of delivery systems possible in larger versions of this invention, the need for compressive force to push the stent means that an over-the-wire system would not generally work in a 1 Fr stent kit, for example. This invention overcomes this area of difficulty.

One delivery system of the stent kit in this invention involves placing a sheath in the ureter by pushing it through the urethra and bladder and into the ureter with a stent inserter or wire guide inside it. The sheath placement allows for the stent to be pushed through the sheath by the inserter, which allows for the stent inserter to be larger than the stent. Having as large a stent inserter as would fit the delivery system is essential, given the dimensions of the component parts in the smaller versions of the stent and the fact that the inserter must be able to push past stones in the ureter, as part of the initial placement of the sheath. It is also essential to use materials of a high compressive strength for the same reason. The design of this invention allows for both necessities.

The stent itself would typically be made from coiled, solid wires for strength and would be coated or plated with a metallic substance, such as gold, for radiopacity and to prevent bacteria adhering to the surface of the stent and making its way from the bladder to the kidney, which could be thus infected. The coils of the stent are close together and would generally be touching adjacent coils, which provides further strength in the stent. This radial strength is needed to prevent deformation by stones or strictures in the ureter.

The coils also provide a further essential function of allowing urine to squeeze into the coil from the ureter or renal pelvis and to drain through the lumen of the stent into the bladder.

The coil continues into a pigtail or loop at the end of the stent, which gives the anchoring anti-migration pigtail or loop more strength. This strength is essential, given that some of the intended uses of this product are in stents of 1 Fr or less in outside diameter to facilitate placement in small animals and for possible pediatric use.

### BRIEF DESCRIPTION OF THE DRAWINGS

FIG. 1a is an illustration of prior art and FIG. 1b is an illustration of prior art;

FIG. 2 is an illustration of the inserter;

FIG. 3a is a cross-sectional view of the stent inserter with the mandrel placed inside it;

FIG. 3b is a cross-sectional view of the stent inserter without any mandrel inside it;

FIG. 4 is an illustration of the outer sheath;

FIG. 5 is an illustration of the radiopaque tip of the sheath and the stent inserter;

FIG. 6 is an illustration of the sheath with the stent inserter inside it;

FIG. 7 is an illustration of the cross-sectional view of the sheath;

FIG. 8 is an illustration of the markings on the proximal end of the inserter;

FIG. 9 is an illustration of a coiled pig-tailed stent;

FIG. 10 is an illustration of a longitudinal section of the coiled stent;

FIG. 11 is an illustration of a coiled stent with a pig-tail on one end only;

FIG. 12 is an illustration of a cross-sectional view of the stent.

### DETAILED DESCRIPTION OF THE INVENTION

One embodiment of the present invention is a kit for placing a stent. This kit will typically include an outer sheath, stent inserter, stent and a tweezers. The sheath and inserter are designed to be attached or detached according to the user requirements and are used solely for placement of the indwelling stent and removed after this has been achieved.

The stent in this embodiment is typically made from metal wires, which have been coiled over a mandrel to create a cylindrical shaped part possessing an internal lumen which makes up the body of the stent. The ends of the stent would typically be formed into a loop or a pigtail at the distal and proximal ends. The wire of the stent will typically be terminated by laser welding the wire into the end to create an atraumatic surface.

There are numerous possible embodiments associated with the stent kit and stent design for the invention described herein and these are detailed in the claims below. All drawings, summaries, descriptions, embodiments and objects are intended to be illustrative rather than limiting.

### Embodiments

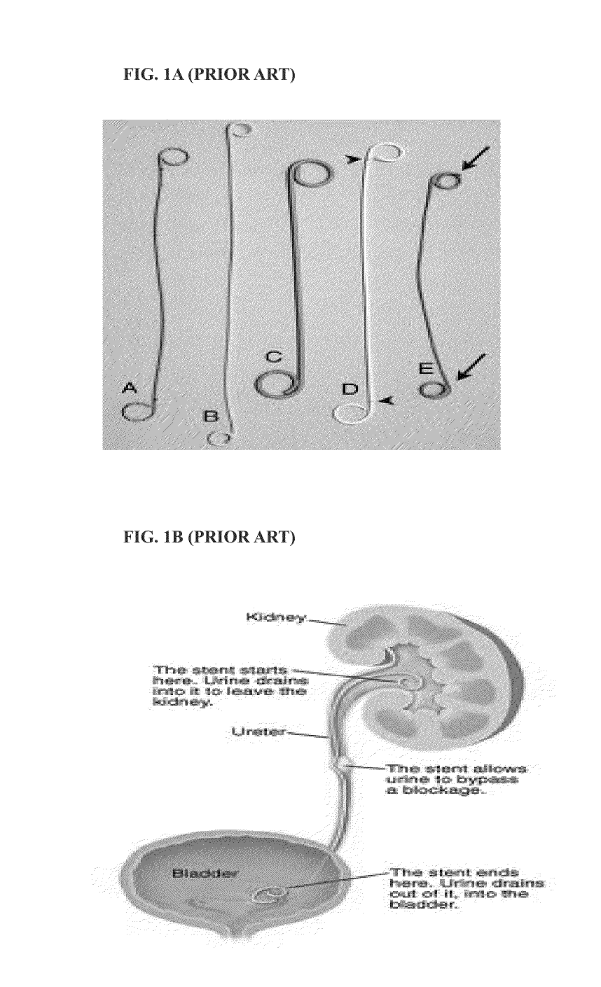

FIG. 1A shows prior art of ureteral stents. FIG. 1B shows the function of ureteral stents.

Typically, these stents are placed in a minimally invasive manner by passing the stent over a guide wire that has been positioned in the renal pelvis of the kidney. A pusher is used to advance the stent along the wire from the urethra to the bladder and subsequently into the ureter.

Other methods for stent placement are percutaneously where the physician accesses the ureter through the skin of patient using nephrostomy methods.

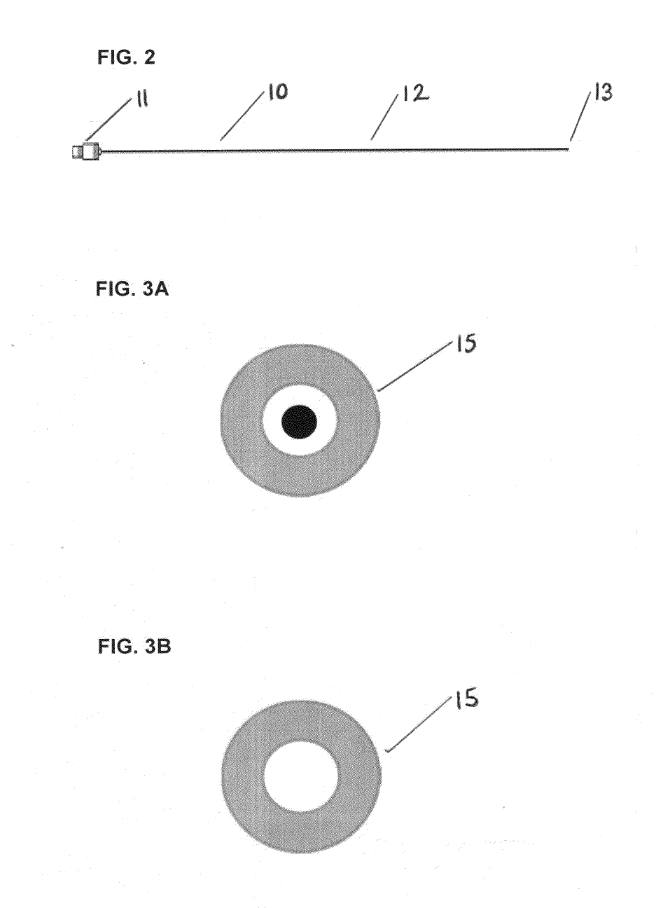

FIG. 2 displays the stent inserter and outer sheath. An embodiment of the design detailed in FIG. 2 is the small diameter of the outer sheath. The stent inserter reference number in FIG. 2 is 10. This significantly reduces trauma to the patient during the procedure and is specifically suitable for patients with ureters that are sized in the range from 0.3-0.7 mm (0.011-0.027″). Other physician benefits are the highly radiopaque properties of the stent and the inserter tip allowing the physician to place the device without the need for a guide wire. This would be of particular benefit for paediatric or small animal veterinary procedures where it is important to keep fluoroscopic exposure to a minimum.

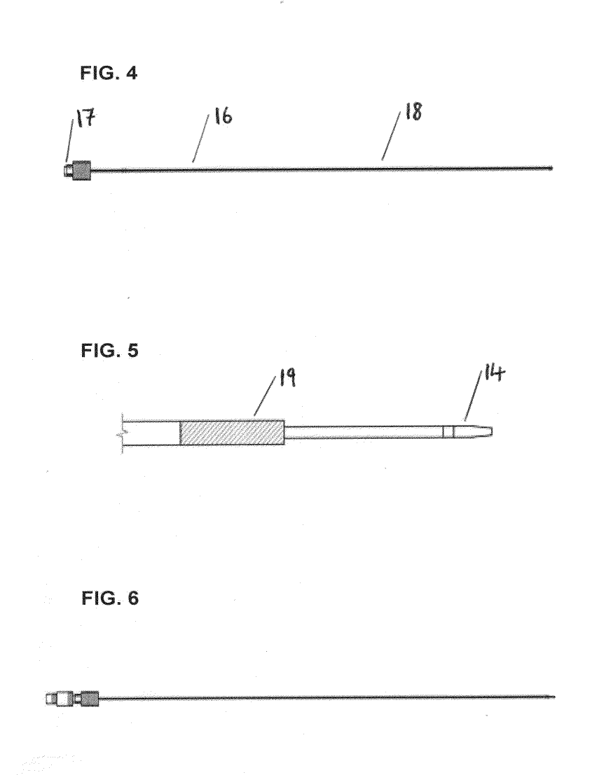

A sheath incorporating one embodiment of the invention is generally indicated by the reference number 16 in FIG. 4.

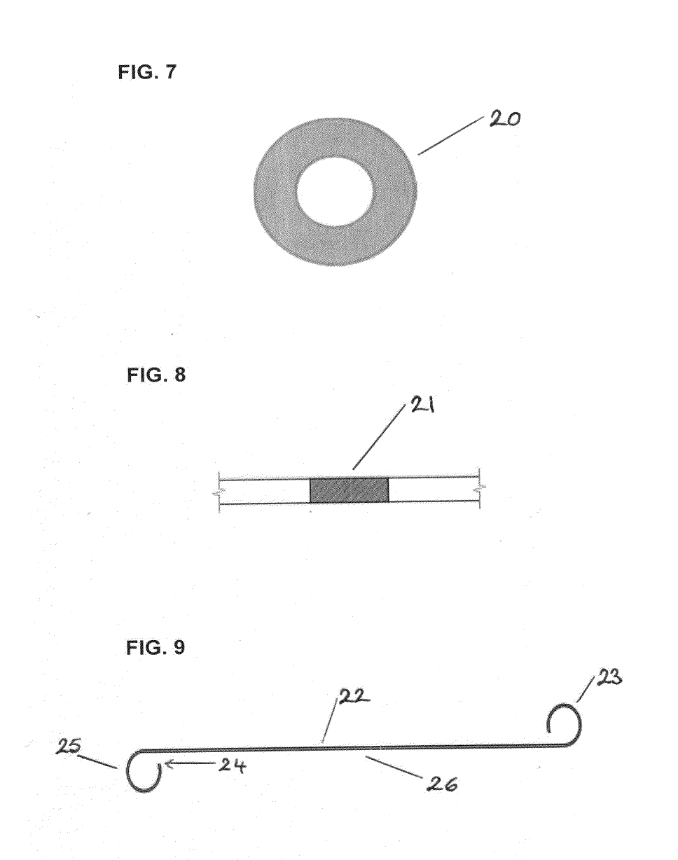

Materials most suitable for the outer sheath might be PTFE, FEP, Fluoropolymer, Silicone, Polyurethane, Polyethylene, Pebax and Nylon, but any material approved for use in medical device can be used. The sheath would typically be flared at the proximal end. The flared end can be attached to the connector cap or handle by gluing or by over-moulding or by welding the proximal end on to a connector cap or handle 16. The connector cap could have a male ending to enable it to attach (FIG. 6) to or detach from the female luer 17 (FIG. 4) of the inserter. The distal end has a radiopaque marker as seen in FIG. 5, reference number 19. The radiopaque marker would typically be a marker band, a radiopaque filler encompassed in the plastic of the sheath or radiopaque ink. The dimensions of the sheath would typically be 15 cm to 60 cm in length 18 (FIG. 4) and typically 0.011″-0.090″ in diameter 20 (FIG. 7).

The inserter 10 (FIG. 2) can consist of a metal or a plastic polymer material such as, but not restricted to PVC, Polyurethane, Polyethylene, Silicone, FEP, Pebax, Polyamide, Polyimide and PEEK. Alternatively, the stent inserter can be a combination of a polymer tube surrounding a metal cannula or mandrel (FIG. 3a). The dimensions of the inserter 10 would typically be 15 cm to 60 cm in length and typically 0.007″-0.090″ in diameter 15 (FIG. 3a & FIG. 3b). The stent inserter could have markings 21 (FIG. 8) on its length 12 (FIG. 2). to denote distance of the distal end of the inserter within the outer sheath and/or to give indication of the stent and/or inserter position during the procedure.

If the inserter was to be made of a cannula or mandrel surrounded by a polymer tube, the metal part may be the same length as the polymer tube 13 or it could be shorter than the tube to allow for a polymer atraumatic tip. Alternatively the metal inserter could be shaped to form an atraumatic tip. The metal inner section may also be ground or electropolished to taper at the distal end to create an atraumatic tip 14 (FIG. 5).

For successful placement of the inserter tip in the renal pelvis of the kidney, it is essential that the distal tip of the inserter is highly radiopaque 14. This can be achieved by: incorporating a radiopaque filler in the distal section of the polymer inserter; a radiopaque metal maker band; radiopaque ink; metal plating with a radiopaque metal; or a radiopaque polymer strip embedded in the distal tip during processing. The metal part of the inserter can be attached to the outer inserter tube via a luer and a connector cap 11. The polymer end of the inserter would typically be flared with a female luer end to allow the physician to attach or detach it from the outer sheath.

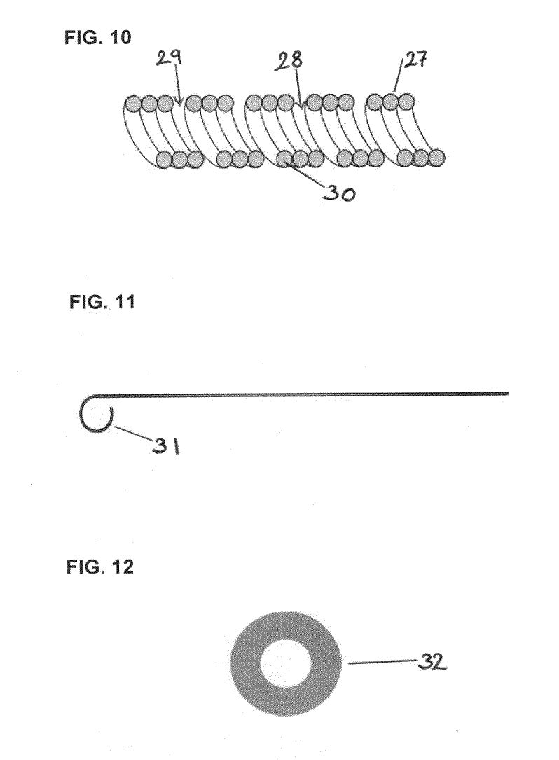

An embodiment of the design detailed in FIG. 9 is a coiled stent 22. The stent can be produced by coiling a metal wire such as stainless steel, Nitinol, MP35N or MP159 or by using a mix of metal and a polymer. The coil pattern can be a any hilar pattern or a variety of hilar patterns 28 (FIG. 10).

Pressure generated in the kidney and ureter would force urine in through the coils 29 (FIG. 10) of the stent and it could drain into the bladder through the coils in the proximal end of the stent.

The stent would typically have a looped pigtail 23 on both the distal and proximal ends to prevent migration of the stent from its position in the ureter. The loops would be created by heat-forming the metal used in the stent. The diameter 25 of the loop/pigtail can be in the range of 3-16 mm.

It is also possible for the stent to have a straight body and only transition into a loop or pigtail on one end 31 (FIG. 11). The stent can also be produced by a braided mesh and with polymer covering/membrane over the mesh .

Other additions to the stent design could consist of the a polymer coating or membrane over the stent. Holes could be created in the membrane of the stent to allow urine to flow into or out of the stent, according to the pressure exerted. The holes could be created by perforating the membrane with a sharp tool or by laser.

The wire used to create the stent would typically range in size from 0.0005″ to 0.040″ 30 (FIG. 10).

The wire can be any produced in various shapes. The diameter of the stent would typically be from 0.3 Fr to 5.0 Fr, as seen at reference 32 (FIG. 12) . The length of the stent would typically range from 3 cm to 20 cm, as seen at reference 26 (FIG. 9).

Alternatively, the stent could be created by laser cutting a hollow tube and forming it into the required shape. The laser cutting could begin with a hypotube, HHS, mandrel or cannula to form the required shape. The thickness of the metal tube used to create the stent could range from 0.0005″ to 0.040″. The laser could cut out a single design iteration or a variety of designs.

The tip of the stent 24 (FIG. 9) could be created by soldering or laser welding the end of the wire into the end of stent or by welding a dome shaped cap on the end of the stent or a plastic or polymer tip. The intention regarding the tip 24 is that it would be atraumatic to the body of the patient. The stent can be plated both on with a metallic material, such as gold, to enhance its radiopaque properties, prevent encrustation and to provide a smooth surface, onto which bacteria cannot easily adhere. Anti-bacterial or anti-encrustation coating may also be applied to the stent's surface 27 (FIG. 10) to prevent or reduce encrustation of the stent for its indwell duration.

What is claimed is:

1. A urology stent made of any number of solid wires coiled together into a helix, with the coil formed into the shape of a pigtail or a partial loop or a single loop or a number of loops at both ends or at one end only and the coiling continuing throughout the length of the stent with a lumen through the centre of the coil.

2. The stent of claim 1, with an atraumatic tip on either end.

3. The stent of claim 1, where a laser-cut hypotube is used instead of a coil.

4. The stent of claim 1, where a hollow wire, with or without holes along its length is used instead of a coil.

5. The stent of claim 1, coated with plastic, such as a polymer, or surrounded with a tube, which may or may not have holes in the tube or plastic coating.

6. The stent of claim 1, where the wires feeding into the anti-migration loop feature at either or both ends of the stent are braided or plaited or welded together or attached to another anti-migration feature.

7. The stent of claim 1, where consecutive coils touch against each other or where hollow wire is used in the coil instead of solid wire.

8. The stent of claim 1, coated or plated with silver, gold or another radiopaque material or coated with an anti-bacterial coating or a drug.

9. The stent of claim 1, where the coil transitions from a coil to an uncoiled section in the body of the stent and that uncoiled section is covered with a tube or is uncovered and is reinforced with a mandrel or a cannula or is not reinforced.

10. A stent kit containing an outer sheath, a stent inserter, incidental parts and the urology stent of claim 1, including the variations to the stent of claim 1, as claimed in claims 2-9.

11. The kit of claim 10, where the outer sheath is a tube with a lumen throughout its length into which the stent will be inserted at the proximal end and pushed along the length of the tube by the stent inserter until it emerges in the renal pelvis and is deployed by pulling back the sheath and stent inserter.

12. The kit of claim 10, where the outer sheath contains a radiopaque marker or multiple radiopaque markers on the distal end of the sheath.

13. The kit of claim 10, where the outer sheath is coated with a hydrophilic coating.

14. The kit of claim 10, where the stent inserter is made of a single material or a composition of materials of compressive strength greater than 50 kPa or braided or coiled materials where the resulting inserter has compressive strength greater than 50 kPa.

15. The kit of claim 10, where the stent inserter may be attached and detached from the outer sheath.

16. The kit of claim 10, where the stent inserter has markings on its length to denote distance from the tip or distal end of the inserter or to give indication of the stent position during the stent insertion procedure.

17. The kit of claim 10, where the tip or distal end of the stent inserter is atraumatic or where the tip or distal end of the stent inserter is radiopaque or has radiopaque marker bands on it.

18. The kit of claim 10, where the stent inserter is surrounded by a tube that can be detachable or not detachable or where any surface of the inserter or sheath is coated with a lubricious coating or a hydrophilic coating.

19. The stent kit of claim 10, where the system of deployment is through pulling back the sheath with the stent inserter held in position until the sheath is pulled back and the proximal end of the stent is fully deployed, at which point the sheath is in line with the inserter and both sheath and inserter can be withdrawn from the body in conjunction with each other or separately.

20. The components of the stent kit of claim 10, where any of the components is used in conjunction with a wire guide, such as an over-the-wire system, a long-wire system or a short-wire system or where the wire exits through a side port.
